(19) United States
(12) Patent Application Publication
(10) Pub. No.: US 2003/0232370 A1
(43) Pub. Date: Dec. 18, 2003

(54) Glucose sensor and uses thereof

(76) Inventor: Mark A. Trifiro, Montreal (CA)

Correspondence Address: Pillsbury Winthrop LLP, Intellectual Property Group, Suite 200, 11682 El Camino Real, San Diego, CA, 92130-2593, US

(21) Appl. No.: 10/421,360

(22) Filed: Apr. 22, 2003

Related U.S. Application Data
(60) Provisional application No. 60/374,126, filed on Apr. 22, 2002.

(51) Int. Cl. C12Q 1/68; C07H 21/04; C12N 9/12; C12P 21/02; C12N 5/06

(52) U.S. Cl. 435/6; 435/69.1; 435/320.1; 435/194; 435/325; 536/23.2

(57) ABSTRACT

The present invention provides a glucokinase protein in which the catalytic activity has been disabled in order to enable its use as a glucose sensor. The catalytically disabled glucokinase protein can be used as the glucose sensor in hand-held glucose monitors and in implantable glucose monitoring devices. The glucose sensor can also be incorporated into biomedical devices for the continuous monitoring of glucose and administration of insulin.

### FIELD OF THE INVENTION

The present invention pertains to the field of glucose sensors, in particular, to a glucokinase protein, wherein the catalytic enzymatic activity has been disabled, yet the protein retains a high specific affinity for and ability to bind glucose.

### BACKGROUND

Glucose control in diabetics is of paramount importance. While poor glucose control leads to morbidity and associated mortality, good glucose control has been shown to reduce cardiovascular, retinal, and kidney diseases by almost 50%, in addition to considerably reducing other complications [The Diabetes Control and Complications Trial Research Group. N. Engl. J. Med. 329:977-986 (1993)].

The push for better management of glucose control in the past led to the development of conventional hand-held glucose monitors. While the use of such glucose monitors has improved insulin strategy, actual insulin delivery remains inflexible, ie. a fixed dose via a systematic route. In contrast, the normal physiological insulin delivery system, the pancreatic islet cells, is a much more sophisticated system that allows perfect glucose control by measuring blood glucose and delivering the appropriate insulin into the portal vein on a minute to minute basis.

In order to provide a more flexible and effective means of insulin delivery, insulin pumps were developed in the 1980's. These pumps allowed an individual to dial in a flexible dosage of insulin and led to the development of implantable insulin devices with large insulin reservoirs that need to be replenished only three to four times a year. Such devices are usually placed in the peritoneum to deliver insulin to the portal venous system and are replenished transdermally. Several hundred devices have been implanted into diabetics to date [Olsen, C. L. et al., Diabetes Care 18:70-76 (1995); Buchwald, et al., ASAIO J. 40:917-918 (1994); Broussolle, C. et al,. Lancet 343:514-515 (1994); Olsen, C. L. et al., Int. J. Artificial Organs 16:847-854 (1993);

Selam, J. L. et al., Diabetes Care 15:877-885 (1992)]. This system of insulin delivery, however, still relies on external monitoring of blood glucose levels and thus has been coined an “open loop system.” The incorporation of an endogenous glucose sensor into this system would render it a “closed loop system” capable of continuous quantitation of glucose and subsequent delivery of an appropriate amount of insulin.

Various methodologies have been employed to create efficient glucose sensors. While glucose sensors have been developed using physical chemical approaches, such sensors tend to lack both specificity and sensitivity. For example, an infrared device has been developed which measures blood glucose, however, this device is reliant on complex computer analysis of the emission spectra to enhance the relatively weak glucose signal and distinguish it from background noise [Robinson, et al., Clin. Chem. 38:1618-1622 (1992.)].

A biological approach to developing glucose sensors offers the advantages of high specificity and sensitivity, and an option of distinguishing different isomers of the same compound. Biological systems are already widely used in clinical chemistry and are also found in all current hand held glucometers, which incorporate the enzyme glucose oxidase into the glucose sensing system.

A number of implantable glucose sensor systems have been proposed. For example, U.S. Pat. Nos. 4,650,547; 4,671,288; 4,781,798; 4,703,756; 4,890,620; 5,569,186 and 5,964,993 all disclose implantable enzyme-based glucose sensors. The glucose sensing ability of these implantable devices, like that in conventional hand-held glucometers, is based on the activity of the enzyme glucose oxidase, which catalyses the oxidation of glucose to yield gluconolactone and hydrogen peroxide. The sensors described in the above-listed patents monitor either the consumption of oxygen or the generation of hydrogen peroxide as an indication of glucose concentration.

A major drawback inherent in these systems is the fact that enzyme-catalysed reactions are greatly affected by the concentration, and therefore the availability, of their reactants. Thus, if access of either glucose or oxygen to the device containing the glucose oxidase is compromised in any way, the results obtained from measuring the catalytic activity of the enzyme will be inaccurate. In the blood, for example, the glucose concentration is typically much higher than the concentration of available oxygen, therefore, the rate of the enzyme-catalysed oxidation of glucose will be controlled by the oxygen concentration and will not accurately reflect the concentration of glucose. In addition, since these devices depend upon the enzyme maintaining its catalytic activity, they must be protected from any molecules, such as inhibitors, that may interfere with this enzyme activity. Furthermore, if the device is monitoring hydrogen peroxide generation, it must also be protected from certain endogenous enzymes, such as catalase, that utilise hydrogen peroxide as a substrate.

An implantable glucose oxidase based biosensor has recently been introduced by Medtronic MiniMed in the U.S. Since this sensor also relies on the catalytic activity of the enzyme glucose oxidase, it is subject to the same drawbacks indicated above. This biosensor has been limited to investigational use only by U.S. law.

Other proteins have been proposed as candidate biosensors for glucose. For example, U.S. Pat. No. 6,197,534 describes engineered proteins for analyte sensing. This patent specifically discloses a glucose/galactose binding protein (GGBP) to which a detectable label has been attached. The detectable quality of the label changes in a concentration-dependent manner upon glucose binding to the protein, thus allowing the presence or concentration of glucose in a sample to be determined. The biosensors described in this patent are proposed for use in hand-held glucometers only.

U.S. Pat. No. 6,277,627 discloses a glucose biosensor comprising a genetically engineered glucose-binding protein (GBP). The GBP is engineered to include mutations that allow the introduction of environmentally sensitive reporter groups the signal from which changes with the amount of glucose bound to the protein. The biosensors described in this patent are proposed for use in the food industry, in clinical chemistry or as part of an implantable device.

While both U.S. Pat. Nos. 6,197,534 and 6,277,627 disclose biosensors to directly measure glucose concentration, which are not reliant upon the catalytic property of an enzyme, they still face certain drawbacks. Of these, the most significant is that both GGBP and GBP, like glucose oxidase, are bacterially derived and are not, therefore, necessarily optimized for detection of physiological concentrations of glucose in a human subject. Both biosensors require incorporation of detectable labels or reporter systems into the protein and the resultant requirement for an appropriate light source for the reporter systems limits the ability of these sensors in an implantable device.

Only a small number of proteins are known that bind glucose. As mentioned above, current protein-based glucose sensors employ bacterially derived proteins, most usually glucose oxidase. Notable drawbacks to the use of this protein include the fact that no known human counterpart exists and thus its use may have unfavourable antigenic consequences. It is also a very large, highly glycosylated protein (186,000 kD), which requires the co-factor flavin mononucleotide for activity. The kinetics of glucose oxidase are unknown and, to date, it has not been cloned.

Known human proteins that bind glucose are either enzymatically active or membrane-bound (ie. insoluble). Amongst the enzymatically active proteins, glucokinase is an exquisitely specific enzyme that binds only the physiological isomer of glucose (D-glucose), and no other sugars, with real affinity (K_{M}=6 mM). Glucokinase belongs to a family of enzymes known as hexokinases. The structure of human brain hexokinase I has been determined by X-ray crystallography [Aleshin, A. E., et al, Structure, 6:39-50 (1998); Aleshin, A. E., et al, J. Mol. Biol., 282:345-357 (1998)]

Human glucokinase is found in only two tissues, the liver and the β-islet cells of the pancreas, where it is believed to be involved in determining levels of insulin secretion. It is a cytoplasmic protein (i.e. soluble) and both liver and pancreatic isoforms have been cloned [Tanizawa, Y., et al., Mol. Endocrinol., 6:1070-1081 (1992); Koranyi, L. I., et al., Diabetes, 41:807-811 (1992); Tanizawa, Y., et al., Proc. Nat. Acad. Sci. USA, 88:7294-7297 (1991)].

Three isoforms of human glucokinase are known: isoform I, specific to islet cells is 465 amino acids in length, and isoforms 2 and 3, specific to liver cells, are 466 and 464 amino acids in length, respectively. The tissue distribution of glucokinase is due to the presence of alternative promoters, which initiate transcription at different loci in the glucokinase gene. These cell-tissue specific promoters dictate very similar cDNAs that differ only at their 5′ ends. Of the 10 exons that make up the cDNA, exons 2-10 are identical in both tissues. However, exon 1 of the transcripts maps to different loci of the glucokinase gene and differs not only in the 5′ untranslated region, but also in the initial 48 nucleotides of the protein coding sequence. Thus the N-terminal ends of the three isoforms of the 52 kD polypeptide differ in their first 14, 15 and 16 amino acids.

Glucokinase catalyses the phosphorylation of glucose to yield glucose-6-phosphate, a reaction that requires ATP as co-substrate. The kinetics of glucokinase activity have been well-studied and demonstrate that binding of glucose to the enzyme occurs independently of ATP binding [Malaisse, W. J., et al., Archives Internationales de Physiologie et de Biochimie, 97:417-425 (1989); Pollard-Knight, D., et al., Biochem. J., 245:625-629 (1987)]. The reaction mechanism is an ordered Bi-Bi sequential mechanism in which the substrate glucose binds first and the product glucose-6-phosphate leaves last.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a glucose sensor comprising a glucokinase protein, wherein the catalytic enzymatic activity has been disabled. The protein retains a high specific affinity for and ability to bind glucose with the appropriate kinetics to be considered as a glucose sensor in a biomedical device.

In accordance with one aspect of the present invention, there is provided a recombinant human glucokinase having decreased catalytic activity but a substantially identical ability to bind glucose relative to the corresponding wild-type human glucokinase.

In accordance with another aspect of the present invention, there is provided an isolated nucleic acid molecule encoding a mutant human glucokinase having decreased catalytic activity but a substantially identical ability to bind glucose relative to the corresponding wild-type human glucokinase.

In accordance with further aspect of the present invention, there are provided vectors comprising an isolated nucleic acid molecule encoding a catalytically disabled human glucokinase and host cells comprising these vectors.

In accordance with another aspect of the invention, there is provided a method of producing a recombinant catalytically disabled human glucokinase comprising culturing a host cell containing a vector encoding the glucokinase under conditions in which the glucokinase is expressed and isolating the expressed glucokinase.

In accordance with another aspect of the invention, there is provided a glucose sensor comprising a recombinant human glucokinase having decreased catalytic activity but a substantially identical ability to bind glucose relative to the corresponding wild-type human glucokinase;.

In accordance with a further aspect of the invention, there is provided a method of determining the level of glucose in a sample comprising contacting the sample with a recombinant catalytically disabled glucokinase, measuring a change in a physical characteristic of said recombinant glucokinase and then correlating this change to the level of glucose in the sample.

### BRIEF DESCRIPTION OF THE FIGURES

FIG. 1 depicts the nucleotide sequence of the cDNA of the liver isoform 2 of glucokinase (GenBank Accession No. M69051).

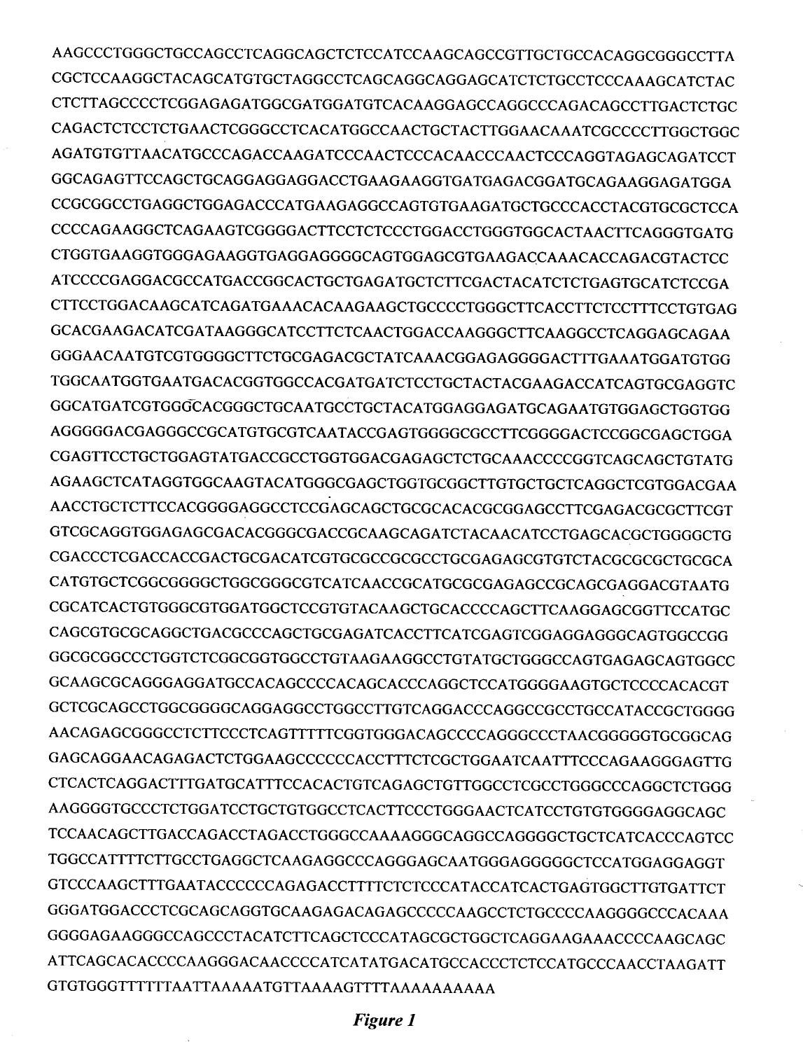

FIG. 2 depicts the amino acid sequence of the cDNA of the liver isoform 2 of glucokinase (GenBank Accession No. AAB59563).

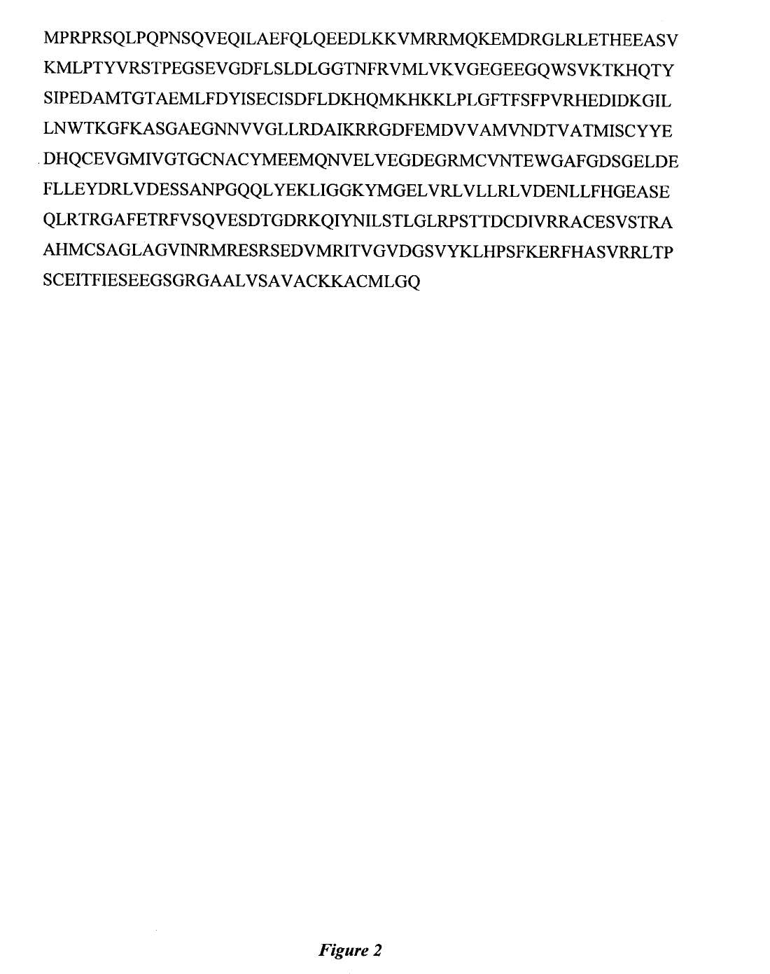

### DETAILED DESCRIPTION OF THE INVENTION

Definitions

Unless defined otherwise, all technical and scientific terms used herein have the same meaning as commonly understood by one of ordinary skill in the art to which this invention pertains.

The term “catalytic activity-disabled” (CAD), as used herein, means that the enzymatic activity of the enzyme (i.e. glucokinase) has been significantly inhibited, such that the glucokinase still binds glucose, but does not catalyze the phosphorylation of glucose to yield glucose-6-phosphate.

The term “CAD-glucokinase,” as used herein, means a glucokinase enzyme in which the catalytic activity has been disabled. In accordance with the present invention, the catalytic activity of the glucokinase enzyme is disabled by genetically engineering one or more appropriate mutation into the enzyme such that the glucokinase still binds glucose, but does not catalyze the phosphorylation of glucose to yield glucose-6-phosphate.

The term “mutation,” as used herein, refers to a deletion, insertion, substitution, inversion, or combinations thereof, of one or more nucleotide in a gene.

Catalytic Activity-disabled Human Glucokinase Protein

The present invention provides a glucokinase protein in which the enzymatic activity has been disabled in order to enable its use as a glucose sensor. In accordance with the present invention, the enzymatic activity of the glucokinase protein has been significantly inhibited, yet the protein retains a high specific affinity for and the ability to bind glucose. In contrast to known glucose sensors, the catalytic activity-disabled glucokinase (CAD-glucokinase) according to the present invention is derived from a human enzyme and thus is naturally optimised to function throughout the normal physiological range of glucose concentrations. Since the binding of glucose to glucokinase has been shown to occur independently of ATP binding, the catalytic activity-disabled human glucokinase does not require any additional substrates or an energy source in order to bind glucose. In addition, the CAD-glucokinase does not rely on a catalytic reaction to determine glucose concentrations.

Glucose sensors based on the CAD-glucokinase according to the present invention can be used in hand-held monitors, in implantable biosensors or can be incorporated into biomedical devices for continuous glucose monitoring and insulin delivery.

The CAD-glucokinase of the present invention is a recombinant glucokinase protein that has been genetically engineered to negate the catalytic activity, but to leave the glucose binding properties of the protein largely intact. As the N-terminal differences of the liver and pancreatic isoforms of glucokinase do not have any demonstrable effect on the functional properties of the protein, the present invention contemplates the use of various isoforms of glucokinase for the generation of a CAD-glucokinase.

Thus, in the context of the present invention, a CAD-glucokinase is provided by introduction of one or more mutation that interferes with the catalytic mechanism of the enzyme and/or interferes with ATP binding. Such effects on the catalytic mechanism or ATP binding can be achieved by deletion and/or substitution of one or more of the amino acids involved, directly or indirectly, in either ATP binding or in catalysis, but not in glucose binding.

As one skilled in the art will appreciate, introduction of a null enzymatic phenotype into the glucokinase creates the potential for ATP binding to the glucokinase to create a ternary complex that may simulate “suicide” or “dead-end” non-competitive inhibition and/or to produce additional conformational changes not related to glucose concentration and/or to interfere with the dissociation of glucose, none of which are desirable in a glucose sensor. The CAD-glucokinase in accordance with one embodiment of the present invention, therefore, is engineered such that the ability to bind ATP is compromised, or abolished. This can be achieved, for example, by mutation of at least one residue involved, directly or indirectly, in ATP binding. Mutation of ATP-binding residues will also help to prevent other related substrates (e.g. inorganic pyrophosphate, PPi) from binding at this site and potentially affecting glucose binding and/or causing conformational change.

Many of the catalytically important amino-acid residues have been identified in glucokinase, as have many of those involved in both glucose and ATP binding. The residues Lys169, Thr 168, Asn231, Asn204, Glu256, and Glu290 have been identified as the main residues constituting the active binding site for glucose in glucokinase [Mahalingam, B., et al., Diabetes, 48:1698-1705 (1999); St. Charles, et al., Diabetes, 43:784-791 (1994); Pilkis, S. J., et al., J. BioL Chem., 269:21925-21928 (1994); Xu, L. Z., et al., J. Biol. Chem., 269:27458-27465 (1994); Lange, A. J., et al., Biochem. J., 277:159-163 (Pt 1) (1991); Takeda, J., et al., J. Biol. Chem., 268:15200-15204 (1993)]. The active amino acids in the ATP-binding cleft include: Gly81, Arg85 and Lys169 (interact with γ-O3 phosphate group); Asp78, Ser151 and Asp205 (interact with Mg²⁺ of Mg-ATP); Thr82, Asn83 and Thr228 (interact with the α-O3 phosphate group); Lys169 (interacts with the β-O3 phosphate group); Ser336 (interacts with the adenine moiety); and Lys296, Thr332 and Ser411 (interact with the ribose moiety). In addition, Asp205 has been identified as the most catalytically important residue, acting as the base catalyst that promotes nucleophilic attack of the 6-hydroxyl group of glucose on the (-phosphate of ATP. Replacement of this residue with alanine has been shown to result in 1,000-fold reduction of enzyme activity, without a significant change in either glucose or ATP binding affinity [Lange, A. J., et al., Biochemical Journal 277 (Pt 1):159-63 (1991)].

Furthermore, natural mutations that occur in glucokinase offer a wealth of information regarding structure-function relationships. Missense mutations linked to early onset non-insulin dependent diabetes mellitus (MODY) have been well characterised [Page, R. C., et al., Diabetic Medicine, 12:209-217 (1995); Xu, L. Z., et al., J. Biol. Chem., 270:9939-9946 (1995); Xu, L. Z., et al., J. Biol. Chem., 269:27458-27465 (1994); Shimokawa, K., et al., J. Clin. Endocrinol. Metab., 79:883-886 (1994); Wajngot, A., et al., Diabetes, 43:1402-1406 (1994); Lange, A. J., et al., Biochem. J., 277:159-163 (Pt 1), (1991); Takeda, J., et al., J. Biol. Chem., 268:15200-15204 (1993); Stoffel, M., et al., Proc. Nat. Acad. Sci., USA, 89:7698-7702 (1992)] and support the roles of some of the above-mentioned residues (e.g. the mutations Glu256Lys and Thr228Met both drastically reduce Vₘₐₓ, with Glu256Lys causing a 3-fold decrease in K_{M} for glucose but Thr228Met leaving the K_{M} for glucose unaffected) as well as providing guidance for the selection of appropriate residues to mutate to produce a CAD-glucokinase. Studies of naturally occurring glucokinase mutations in MODY patients have indicated that Val203 and Gly261 residues are important in a glucose induced fit effect and ATP binding, respectively [Liang, Y, et al., Biochem. J., 309:167-173 (1995)].

Provided with the structure/function information available for glucokinase, one skilled in the art can readily select appropriate amino acids for mutation in engineering a CAD-glucokinase. For example, as indicated above, introduction of a mutation at residue 205 vastly decreases the catalytic efficiency of the enzyme and mutation of one of Asp78, Gly80, Thr209, Gly227, Thr228, Ser336, Gly410, Ser411 or Lys414 has the potential to impact the ATP binding ability of the glucokinase. Thus, the present invention contemplates genetically engineered glucokinase proteins in which one or more of the above-mentioned residues involved in catalysis or ATP binding, but not in glucose binding, is altered to produce a CAD-glucokinase that retains its ability to bind glucose. The present invention also contemplates the mutation of residues that are not directly involved in catalysis or ATP binding, but which are in close proximity to residues that are and which may thereby indirectly affect catalysis or ATP binding.

As an alternative to rational selection of appropriate residues for mutation, a random approach to generating mutations in the glucokinase can be adopted using techniques known in the art. The resultant mutants can be screened for their ability to bind glucose and the loss of their ability to catalyse the conversion of glucose to glucose-6-phosphate, thereby isolating CAD-glucokinases in accordance with the present invention.

In one embodiment of the present invention, the genetically engineered CAD-glucokinase is mutated at one or more of the residues Asp205, Ser336, Lys414, Thr228 and Val226. In another embodiment, the CAD-glucokinase contains a mutation at residue Asp205 in combination with a mutation at one or more of residues Ser336, Lys414, Thr228 and Val226. In another embodiment, the CAD-glucokinase contains a mutation at residue Asp205 and at residue Ser336. In another embodiment, the CAD-glucokinase contains the mutation Asp205Ala. In still another embodiment, the CAD-glucokinase contains the mutation Asp205Ala in combination with Ser336Leu; Ser336Val or Ser336Ile. In a further embodiment, the CAD-glucokinase contains the mutation Asp205Ala in combination with Lys414Glu.

Means of Disabling the Enzymatic Activity

As is known in the art, genetic engineering of a protein generally requires that the nucleic acid encoding the protein first be isolated and cloned. Sequences for various pancreatic forms of human glucokinase are available from GenBank (for example, Accession Nos. AAA52562; AAA51824; NP_{—}000153 [protein] and M90299; M88011; NM_{—}000162 [nucleotide]), as is the sequence for the liver isoform (Accession No. AAB59563 [protein], M69051 [nucleotide]). Isolation and cloning of the nucleic acid sequence encoding the human glucokinase can thus be achieved using standard techniques [see, for example, Ausubel et al., Current Protocols in Molecular Biology, Wiley & Sons, NY (1997 and updates); Sambrook et al., Molecular Cloning: A Laboratory Manual, Cold-Spring Harbor Press, NY (2001)]. For example, the nucleic acid sequence can be obtained directly from a suitable human tissue, such as liver or pancreatic tissue or an insulinoma, by extracting the mRNA by standard techniques and then synthesizing cDNA from the mRNA template (for example, by RT-PCR). Alternatively, the nucleic acid sequence encoding human glucokinase can be obtained from an appropriate human cDNA library by standard procedures. The isolated cDNA is then inserted into a suitable vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses. The vector may be a cloning vector or it may be an expression vector. Procedures for cloning human glucokinase are also described in the literature [Koranyi, L. I., et al., Diabetes, 41:807-811 (1992); Tanizawa, Y., et al., Proc. Nat. Acad. Sci., USA, 88:7294-7297 (1991)]. Alternatively, the cloned human pancreatic glucokinase coding sequence can be obtained from the American Type Culture Collection (ATCC) (see ATCC No. 79040 or 79041), as can the cloned glucokinase coding sequence isolated from liver carcinoma (see ATCC No. MGC-1742).

The present invention contemplates the use of one of the known isoforms of glucokinase in the creation of a genetically engineered, CAD-glucokinase as well as those isoforms that may be identified in the future. As mentioned previously, the difference between the cDNA of the liver and the pancreatic isoforms of glucokinase is only at the 5′ end of the cDNA. Therefore, one skilled in the art will appreciate that, once the cDNA of one isoform has been cloned, other isoforms can be readily engineered by addition and/or deletion of the appropriate nucleotides using standard molecular biological techniques.

In one embodiment of the present invention, the CAD-glucokinase is produced from one of the human liver glucokinase isoforms. In another embodiment, the CAD-glucokinase is produced from human liver glucokinase isoform 2. In another embodiment, the CAD-glucokinase is produced from the human pancreatic glucokinase isoform.

Once the nucleic acid sequence encoding human glucokinase has been obtained, mutations can be introduced at specific, pre-selected locations by in vitro site-directed mutagenesis techniques well-known in the art. Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

If desired, after introduction of the appropriate mutation or mutations, the nucleic acid sequence encoding human glucokinase can be inserted into a suitable expression vector. Examples of suitable expression vectors include, but are not limited to, plasmids, phagemids, cosmids, bacteriophages, baculoviruses and retroviruses, and DNA viruses. In one embodiment of the present invention, the nucleic acid encoding the genetically engineered glucokinase is cloned into a baculovirus plasmid.

One skilled in the art will understand that the expression vector may further include regulatory elements, such as transcriptional elements, required for efficient transcription of the glucokinase coding sequences. Examples of regulatory elements that can be incorporated into the vector include, but are not limited to, promoters, enhancers, terminators, and polyadenylation signals. The present invention, therefore, provides vectors comprising a regulatory element operatively linked to a nucleic acid sequence encoding a genetically engineered, CAD-glucokinase. One skilled in the art will appreciate that selection of suitable regulatory elements is dependent on the host cell chosen for expression of the genetically engineered glucokinase and that such regulatory elements may be derived from a variety of sources, including bacterial, fungal, viral, mammalian or insect genes.

In the context of the present invention, the expression vector may additionally contain heterologous nucleic acid sequences that facilitate the purification of the expressed glucokinase. Examples of such heterologous nucleic acid sequences include, but are not limited to, affinity tags such as metal-affinity tags, histidine tags, avidin/strepavidin encoding sequences, glutathione-S-transferase (GST) encoding sequences and biotin encoding sequences.

The expression vectors can be introduced into a suitable host cell or tissue by one of a variety of methods known in the art. Such methods can be found generally described in Ausubel et al., Current Protocols in Molecular Biology, Wiley & Sons, NY (1997 and updates); Sambrook et al., Molecular Cloning: A Laboratory Manual, Cold-Spring Harbor Press, NY (2001) and include, for example, stable or transient transfection, lipofection, electroporation, and infection with recombinant viral vectors. One skilled in the art will understand that selection of the appropriate host cell for expression of the genetically engineered glucokinase will be dependent upon the vector chosen. Examples of host cells include, but are not limited to, bacterial, yeast, insect, plant and mammalian cells.

Methods of cloning and expressing proteins are well-known in the art, detailed descriptions of techniques and systems for the expression of recombinant proteins can be found, for example, in Current Protocols in Protein Science (Coligan, J. E., et al., Wiley & Sons, New York).

The CAD-glucokinase can be purified from the host cells by standard techniques known in the art. If desired, the changes in amino acid sequence engineered into the protein can be determined by standard peptide sequencing techniques using either the intact protein or proteolytic fragments thereof.

As an alternative to a directed approach to introducing mutations into glucokinase, a cloned glucokinase gene can be subjected to random mutagenesis by techniques known in the art. Subsequent expression and screening of the mutant forms of the enzyme thus generated would allow the identification and isolation of CAD-glucokinases.

The present invention also contemplates fragments of the CAD-glucokinase, for example, fragments that comprise the glucose binding domain, which retain the ability to bind glucose but do not catalyse its conversion to glucose-6-phosphate. Such fragments can be readily generated for example, by cloning a fragment of the gene encoding the full-length CAD-glucokinase. Fusion proteins comprising a fragment of a CAD-glucokinase and a heterologous amino acid sequence are also contemplated. Examples of such heterologous amino acid sequences include those encoding an affinity tag, epitope, marker, reporter protein, or the like.

The present invention, therefore, provides isolated nucleic acid molecules encoding a CAD-glucokinase, or a fragment or domain thereof, vectors comprising such nucleic acids as well as host cells comprising the vectors.

Functional Criteria of the Catalytic Activity-disabled Glucokinase

In the context of the present invention, to be useful as a glucose sensor the catalytic activity of the glucokinase is disabled (i.e. the protein does not exhibit significant catalytic activity with respect to the conversion of glucose to glucose-6-phosphate), yet the glucokinase retains the ability to specifically bind glucose with an affinity approaching that of the wild-type enzyme and optionally has significantly reduced or abolished ability to bind ATP.

I. Catalytic Activity

The catalytic activity of the CAD-glucokinase is determined by measuring the ability of the protein to catalyse the phosphorylation of glucose in the presence of ATP. The extent to which the catalytic activity of the CAD-glucokinase has been impaired is then determined by comparison of the measured activity to that of the wild-type enzyme.

Methods of assaying the catalytic activity of hexokinases are known in the art. Assays to measure the activity of glucokinase can be generally based on that described by Storer [Storer, A. C., et al., Biochem. J., 141:205-209 (1974)] which utilises a coupled enzymatic assay employing glucose-6-phosphate dehydrogenase leading to the production of NADPH. The amount of NADPH produced in the assay can readily be measured by monitoring the increase in absorbance at 340 nm. One skilled in the art will appreciate that modifications can be made to the basic assay if desired (for example, see Trifiro, M., et al., Prep. Biochem 16:155-173 (1986)].

In general, preparations of the wild-type or CAD-glucokinase are added to a buffered reaction mixture containing NADP, potassium chloride, glucose-6-phosphate dehydrogenase, glucose and ATP. Phosphorylation of the glucose to glucose-6-phosphate by the glucokinase and subsequent reduction of glucose-6-phosphate and production of NADPH by the glucose-6-phosphate dehydrogenase leads to an increase in absorbance at 340 nm, which is monitored as an indication of the amount of NADPH produced. This value can then be correlated to the activity of the glucokinase or CAD-glucokinase by standard methods.

Glucokinase activity is generally defined in units per millilitre, where one unit of activity is the amount of enzyme that transforms, under optimal conditions, 1 μmole of substrate/min at room temperature. In the context of the present invention a CAD-glucokinase protein is one that has an activity that is between 10 and 10 000-fold less than that of the wild-type enzyme. In one embodiment of the present invention, the activity of the CAD-glucokinase is decreased by between 100 and 10 000-fold when compared to the wild-type enzyme. In another embodiment, the activity of the CAD-glucokinase is decreased by at least 1 000-fold when compared to the activity of the wild-type enzyme.

II. Binding Affinity for Glucose and ATP

The ability of the CAD-glucokinase to bind glucose with an affinity approaching that of the wild-type enzyme is essential. A CAD-glucokinase with an impaired ability to bind glucose will be unable to function efficiently as a glucose sensor.

The binding affinity of the CAD-glucokinase for glucose and ATP can be determined by techniques well-known in the art. The measured binding affinities can then be compared to those of the wild-type enzyme to provide an indication of the extent to which the binding affinities have been affected. Methods of measuring binding affinities are known in the art [for example, see Liang, Y., et al.., Biochem. J., 309:167-173(1995); Shkolny, D. L., et al., J. Clin. Endocrinol. Metab., 84:805-810 (1999)]. In general, the appropriate substrate (i.e. glucose or ATP) is first labelled with a detectable label. The wild-type glucokinase or CAD-glucokinase is then mixed with various concentrations of the labelled substrate and the amount of bound substrate is determined. Results are analysed by standard methods, for example through the use of Scatchard plots, and the binding affinities of the wild-type enzyme and the CAD-glucokinase are compared.

Detectable labels are moieties a property or characteristic of which can be detected directly or indirectly. One skilled in the art will appreciate that the detectable label is chosen such that it does not affect the ability of the wild-type protein to bind the substrate. Labels suitable for use with the substrates include, but are not limited to, radioisotopes, fluorophores, chemiluminophores, colloidal particles, fluorescent microparticles and the like. Examples of suitable labelled substrates include, but are not limited to, trinitrophenyl (TNP)-ATP (Molecular Probes, Eugene, Oreg.), D-glucose 2-³H (NEN, Boston, Mass.) and ³²P α-ATP (NEN, Boston, Mass.). One skilled in the art will understand that these labels may require additional components, such as triggering reagents, light, and the like to enable detection of the label. In one embodiment of the present invention, the substrates are labelled with a radioisotope. In another embodiment, the substrates are labelled with the radioisotope ³H.

In accordance with the present invention, the CAD-glucokinase retains at least 10% of the binding affinity for glucose that is measured for the wild-type enzyme. In one embodiment, the CAD-glucokinase retains at least 20% of the wild-type binding affinity for glucose. In another embodiment, the CAD-glucokinase retains at least 30% of the wild-type binding affinity for glucose. In other embodiments, the CAD-glucokinase retains at least 40% and at least 50% of the wild-type binding affinity for glucose.

In one embodiment of the present invention, the ability of the CAD-glucokinase to bind ATP is either abolished or impaired. Since it has been demonstrated that ATP binding is not required in order for glucokinase to bind glucose, disabling the ATP binding ability of the protein by site-directed mutagenesis will prevent the enzyme from completing the phosphorylation reaction and will thus contribute to its lack of enzymatic activity, but will not interfere with the glucose-binding ability of the protein. In addition, removal of the ATP-binding ability will help to prevent the formation of any dead-end ternary complexes by the protein.

In accordance with one embodiment of the present invention, therefore, the CAD-glucokinase has less than 50% of the binding affinity for ATP that is measured for the wild-type enzyme. In one embodiment, the CAD-glucokinase has less than 40% of the wild-type binding affinity for ATP. In other embodiments, the CAD-glucokinase retains less than 30%, less than 20% and less than 10% of the wild-type binding affinity for ATP.

III. Dissociation Parameters

The ability of the CAD-glucokinase to release glucose or allow glucose to dissociate in a specific time frame is an important issue. If the CAD-glucokinase forms long-lasting glucose-glucokinase complexes, then its ability to sense changing glucose concentrations in relatively short time frames will be jeopardized.

Measurement of parameters such as the dissociation rate (k) for glucose or the half-lives (t_{1/2}, i.e. the time required for 50% of bound glucose to dissociate) of glucose-glucokinase complexes provides an indication of the ability of the CAD-glucokinase to release glucose. Comparison of the value of these parameters with those for the wild-type glucokinase indicates whether this ability is impaired. Determination of the above parameters can be readily achieved by a worker skilled in the art using standard techniques [for example, see Shkolny, D. L., et al., J. Clin. Endocrinol. Metab., 84:805-810 (1999)].

For example, the dissociation rate of a substrate or ligand can be measured by standard dissociation binding experiments using a labelled substrate/ligand. In general, the protein and the labelled substrate are allowed to bind, usually to equilibrium, and then further binding of the labelled substrate is blocked. The rate of dissociation of the labelled substrate from the protein is measured by determining how much substrate remains bound at various time points subsequent to the blocking step. Further binding of the labelled substrate can be blocked by a number of methods, for example, the protein can be attached to a suitable surface and the buffer containing the labelled substrate can be removed and replaced with fresh buffer without labelled substrate. Alternatively, a very high concentration of unlabelled substrate can be added, the high concentration of unlabelled substrate ensures that it instantly binds to nearly all the unbound protein molecules and thus blocks binding of the labelled substrate, or the suspension can be diluted by a large factor, for example 20- to 100-fold, to greatly reduce the concentration of labelled substrate such that any new binding of labelled substrate by the protein will be negligible.

In one embodiment of the present invention, the dissociation constants are determined using glucose radiolabelled with ³H as the substrate and addition of “cold” glucose is used to block further binding of the radiolabelled glucose. At various times, aliquots are removed and the amount of bound and free ³H-glucose is determined.

Rates of dissociation are generally expressed as the fraction of complexes dissociating per unit time and as half-lives of complexes. In accordance with the present invention, the dissociation rate for the CAD-glucokinase is in the order of minutes. In one embodiment, the dissociation rate is 0.1 to 10 minutes (e.g. k=0.1/min to k=0.9/min).

One skilled in the art will appreciate that dissociation kinetics can also be measured in real time using surface plasmon resonance (for example, using BIACORE® technology; Biacore International AB, Uppsala, Sweden). As is known in the art, surface plasmon resonance (SPR) occurs when surface plasmon waves are excited at a metal/liquid interface and enables the monitoring of binding events between two or more molecules in real time. Light is directed at, and reflected from, the side of a surface that is not in contact with a sample and, at a specific combination of wavelength and angle, SPR causes a reduction in the reflected light intensity. Biomolecular binding events cause changes in the refractive index at the surface layer, which are detected as changes in the SPR signal. Advantages to measuring real-time dissociation kinetics include the ability to confirm classical dissociation kinetics and as well as providing real-time kinetic information that is important in establishing the suitability of a CAD-glucokinase as a potential glucosensor [see, Malmqvist, M., Biochem. Soc. Trans., 27:335-339 (1999)].

Use of the Catalytic Activity-disabled Glucokinase as a Glucose Sensor

In accordance with the present invention, the CAD-glucokinase can be used as a glucose sensor, for example, in a hand-held or an implantable glucose-sensing device. The CAD-glucokinase is also suitable for use as the glucose sensor in biomedical devices designed to continuously monitor blood glucose levels and administer insulin.

To function effectively as a glucose sensor, the CAD-glucokinase according to the present invention must possess a measurable characteristic which allows free protein to be distinguished from the glucose-bound protein. Associated with this characteristic, there must additionally be a detectable quality that changes in a concentration-dependent manner when the protein is bound to glucose. An example of one such characteristic is the conformational change that occurs when glucokinase binds glucose.

Conformational Analysis of the CAD-glucokinase

In one embodiment, the present invention takes advantage of the change in conformation which occurs when glucose binds to glucokinase [Gidh-Jain, M., et al., Proc. Natl. Acad. Sci., USA, 90:1932-1936 (1993); Lin, S. X., et al., J. Biol. Chem., 265:9670-9675 (1990); Neet, K. E., et al., Biochemistry, 29:770-777 (1990); Steitz, T. A., et al., Phil. Trans. Royal Soc. London- Series B: Biological Sciences, 293:43-52 (1981); Pickover, C. A., et al., J. Biol. Chem., 254:11323-11329 (1979); McDonald, R. C., et al., Biochemistry, 18:338-342 (1979); Olvarria, J. M., et al., Archivos de Biologia y Medicina Experimentales, 18: 85-292 (1985); Xu, L. Z., et al., Biochemistry, 34:6083-6092 (1995)]. Such a change in conformation is measurable and thus provides a characteristic that will allow free glucokinase and glucose-glucokinase complexes to be distinguished. Conformational changes of proteins have been demonstrated as a basis for biosensing [Wilner B., Nature Biotech., 19:1023-1024 (2001); Benson D. E., et al., Science, 293:1641-1644 (2001)].

The ability of the CAD-glucokinase to undergo a similar conformational change to the wild-type enzyme upon glucose binding can be confirmed by a number of techniques known in the art. For example, partial proteolytic digestion can be used to indicate the folded state of a protein. As is known in the art, any given protease exhibits a certain bond specificity and thus, when used to digest an unfolded protein, will yield a defined set of peptide fragments which can be separated and analyzed, for example by denaturing polyacrylamide gel electrophoresis (PAGE). However, when the treated protein is in a folded or native state, many of the susceptible bonds may be buried within the hydrophobic core of the protein and thus be inaccessible to the protease. The conformational state of the protein, therefore, defines which bonds will be cleaved and consequently, the pattern of peptide fragments produced. Areas most likely to contain susceptible bonds are exposed loops within domains or the linking regions between domains. These accessible regions could be constantly present, or could arise transiently as a result of the protein undergoing a conformational change.

Partial proteolytic digestion has been used to document successfully several protein conformational states and/or changes in conformation [Inoue, S., et al., J. Biochem., 118:650-657 (1995); Hockerman, G. H., et al., Mol. Pharmacol., 49:1021-1032 (1996); Chen, G. C., et al., J. Biol. Chem., 269:29121-29128 (1994)]. More recently, partial proteolytic digestion has been used to document ligand-induced conformation change of several steroid receptors [Couette, B., et al., Biochem. J., 315:421-427 (1996); Kuil, C. W., et al., J. Biol. Chem., 270:27569-27576 (1995); Kuil, C., W., Mulder, E., Mol. Cell. Endocrinol., 102:R1-R5 (1994); Keidel, S., et al., Mol. Cell. Biol., 14:287-298 (1994); Leng, X., et al., J. Steroid Biochem. Mol. Biol., 46:643-661 (1993); Allan, G. F., et al., J. Biol. Chem., 267:19513-19520(1992); Kallio, P. J., et al., Endocrinology, 134:998-1001 (1994)] (100-106).

Partial protease digestion and analysis of resultant peptide fragments, therefore, can be used to demonstrate the conformational change of wild-type glucokinase induced by glucose binding. Once the peptide fragment patterns have been determined for the wild-type glucokinase with and without bound glucose, the peptide fragments generated by partial proteolytic digestion of a CAD-glucokinase protein can then be analysed to determine whether these proteins undergo a similar conformational change. CAD-glucokinase proteins that mimic the conformational changes seen in the wild-type glucokinase can thereby be selected.

Alternatively, a similar technique known as zero order cross-linking can be used. This technique relies on the activity of the enzyme transglutaminase to cross-link lysine and glutamine residues in the protein that are close together in three-dimensional space. Lysine and glutamine residues that are spatially separated will not be affected by the activity of this enzyme. Pre-treatment of a protein with transglutaminase followed by complete digestion with a protease, such as trypsin, thus yields a “fingerprint” of peptide fragments that can be resolved by standard techniques such as denaturing PAGE (see, for example, Safer, D., et al., Biochemistry, 36:5806-5816 (1997)]. Zero-order cross-linking, therefore, can be used to determine the digestion pattern of wild-type glucokinase with or without bound glucose. The pattern of peptides produced from digestion of the CAD-glucokinase proteins pre-treated with transglutaminase can be compared to those of the wild-type protein and those proteins displaying proteolytic peptide fragment patterns similar to those of the wild-type protein can be selected.

A further method that can be used to determine conformational change in the wild-type and catalytic activity-disabled proteins makes use of the redistribution of surface electrical charges that result from large conformational changes in proteins. As is known in the art, most proteins possess a net electrical charge or dipole. Movement of the protein, for example, as the result of binding a substrate, inhibitor or activator, can lead to a change in the overall dipole of the protein, which can be reflected by measurement of simple electrical parameters [see, for example, Mi, L. Z., et al., Biophys. J., 73:446-451 (1997)]. Dielectric relaxation spectroscopy is a standard method of determining dielectric properties of proteins [see, Biophysical Chemistry, Chapter 14E and F, ed. Marshall Allan G, John Wiley & Sons, Inc. NY. (1978)]. In one embodiment of the present invention, dielectric relaxation spectroscopy employing frequency domain or time domain methodology, such as that described by Smith [Smith, G., et al., J. Pharm. Sci., 84:1029 1044 (1995)], is used to determine the dielectric properties and, therefore, the dipole of the wild-type and CAD-glucokinase.

In addition, the use of newer methods such as NMR and X-ray databases [see, for example, Takashima, S., Biopolymers, 54:398-409 (2001)] to determine the dipole of the wild-type and CAD-glucokinase is also contemplated by the present invention.

Alternatively, the conformational change induced by glucose binding to the wild-type and CAD-glucokinase proteins could be compared using BIACORE® technology (Biacore International AB, Uppsala, Sweden), which uses surface plasmon resonance (SPR) as described previously with respect to the measurement of binding affinities for the CAD-glucokinase.

In order to determine conformational changes in the glucokinase protein upon glucose binding using BIACORE® technology, the protein is first immobilized on a sensor surface. This sensor surface forms one wall of a flow cell and a solution containing glucose is injected over this surface in a precisely controlled flow. Fixed wavelength light is directed at the sensor surface and binding events are detected as changes in the particular angle where SPR creates extinction of light. This change is measured continuously and recorded as a sensorgram. After injection of the glucose-containing solution, a continuous flow of buffer is passed over the surface and the dissociation of the glucose from the glucokinase molecule can be determined. The present invention therefore contemplates the use of BIACORE® technology to determine conformational changes in the catalytic activity-disabled proteins, as well as their binding affinity for glucose and dissociation parameters.

BIACORE® technology is known in the art, as are methods of immobilizing proteins on inert surfaces. Appropriate sensor chips for use in these techniques are commercially available from Biacore International AB (Uppsala, Sweden).

Conformational changes can also be determined in proteins through the use of reporter groups. In one embodiment of the present invention, one or more reporter groups are associated with the CAD-glucokinase. The reporter group can be covalently or non-covalently associated with the protein. Glucokinase proteins that have been further genetically engineered to allow incorporation of a reporter group, for example by inclusion of one or more cysteine residues to provide reactive thiol groups are, therefore, also considered to be within the scope of the present invention. In accordance with the present invention, the reporter group is incorporated into the protein such that it produces a detectable signal when the protein undergoes a conformational change.

One skilled in the art will understand that a variety of reporter groups are available and are suitable for use in the present invention. These reporter groups differ in the physical nature of signal transduction (e.g., fluorescence, electrochemical, nuclear magnetic resonance (NMR), or electron paramagnetic resonance (EPR)) and in the chemical nature of the reporter group. Examples of suitable reporter groups include, but are not limited to, fluorescent reporter groups, non-fluorescent energy transfer acceptors, and the like. Alternatively, the reporter may comprise an energy donor moiety and an energy acceptor moiety, each bound to the glucokinase protein and spaced such that there is a change in the detectable signal when the glucokinase is bound to glucose.

When the glucose sensor comprising the CAD-glucokinase is to be incorporated into an implantable device, fluorophores that operate at long excitation and emission wavelengths (e.g., >600 nm) are most useful (human skin being opaque below 600 nm). Presently, there are only a few environmentally sensitive probes available in this region of the spectrum, although others are likely to be developed in the future that are also suitable for use in the present invention. Examples of those available include, thiol-reactive derivatives of osmium (II) bisbipyridyl complexes and of the dye Nile Blue [Geren, et al., Biochem., 30:9450 (1991)]. Osmium (II) bisbipyridyl complexes have absorbances at wavelengths longer than 600 nm with emission maxima in the 700 to 800 nm region [Demas, et al., Anal. Chem., 63:829A (1991)] and long life-times (in the 100 nsec range), simplifying the fluorescence life-time instrumentation. The present invention further contemplates the use of redox cofactors as reporter groups, e.g., ferrocene and thiol-reactive derivatives thereof. Thiol-reactive derivatives of organic free radicals such as 2,2,6,6-tetramethyl- 1-piperinoxidy (TEMPO) and 2,2,5,5-tetramethyl- 1-piperidinyloxy (PROXYL) can also be used and changes in the EPR spectra of these probes in response to ligand binding can be monitored.

Incorporation of the Catalytic Activity-disabled Glucokinase within a Biosensor

Conformational changes induced by ligand binding, such as those induced by glucose binding to glucokinase, have been measured by impedance biosensors (for review, see Berggen et al., Electroanal., 13:173-180 (2001)]. Impedimetric detection works by measuring the impedance changes produced by binding of target molecules to receptor molecules immobilised on the surface of microelectrodes.

In the context of the present invention, a microelectrode consists of a multilayer substrate comprising a conductive base layer and an optional self-assembled monolayer (or other chemical entity) directly or indirectly bound to the conductive base layer. Various conducting or semiconducting substances are known in the art and are suitable for use as the conductive base layer of the microelectrode. Examples include, but are not limited to, gold, silver, and copper (which bind thiol, sulphide or disulphide functional compounds), silicon (either SiH surface which binds alcohols and carboxylic acids, or SiO₂ surface which binds silicon-based compounds such as trichlorosilanes), aluminium, platinum, iridium, palladium, rhodium, mercury, osmium, ruthenium, gallium arsenide, indium phosphide, and mercury cadmium telluride. Examples of suitable forms include foils (such as aluminium foil), wires, wafers (such as doped silicon wafers), chips, semiconductor devices and coatings (such as silver and gold coatings) deposited by known deposition processes.

Self-assembled monolayers (SAMs) are also known in the art and are generally defined as a type of molecule that can bind or interact spontaneously or otherwise with a metal, metal oxide, glass, quartz or modified polymer surface in order to form a chemisorbed monolayer. A self-assembled monolayer should be the thickness of a single molecule (ie., it is ideally no thicker than the length of the longest molecule included therein). Each of the molecules making up a self-assembled monolayer thus includes a reactive group that adheres to the conductive base layer and may also include a second reactive moiety that can be used to immobilize the protein onto the microelectrode. The microelectrode can alternatively be constructed without the use of SAMs (i.e., by direct physical absorption of the protein onto the conductive layer).

The present invention, therefore, contemplates the immobilization of the CAD-glucokinase onto a microelectrode for use as an impedance biosensor. Methods of immobilizing proteins are well-known in the art (for general techniques, see for example, Coligan et al., Current Protocols in Protein Science, Wiley & Sons, NY). Such immobilization generally makes use of reactive groups on the surface to which the protein is to be attached and/or coupling reagents, such as carbodiimide, succinimides, thionyl chloride, p-nitrophenol, glutaraldehyde, cyanuric chloride and phenyl diisocyanate. One skilled in the art will understand that when a coupling reagent is used, its selection is dependent on the chemical nature of the group on the surface to which the protein is to be immobilized.

The present invention also contemplates the use of CAD-glucokinase proteins which have been further engineered to incorporate a group or molecule that facilitates immobilization of the protein to a solid surface. Examples of such groups or molecules include, but are not limited to, hexa-histidine tags allowing immobilization onto Ni²⁺-containing surfaces, arsenic or other metal-binding motifs to allow immobilization onto a surface containing the cognate metal, glutathione-S-transferase fusions that allow immobilisation onto glutathione-containing surfaces, avidin or biotin tags and the like. Thus, CAD-glucokinase proteins engineered to incorporate a group or molecule that facilitates immobilization of the protein are considered to be within the scope of the present invention. One skilled in the art will appreciate that such a group or molecule should not interfere with the binding of glucose by the CAD-glucokinase.

Various biosensors suitable for impedimetric-based sensing have been described in the art. For example, an immunobiosensor has been developed to measure staphylococcus enterotoxin B [DeSilva, M. S., et al., Biosensors & Bioelectronics, 10:675-682 (1995)]. This biosensor contains staphylococcus enterotoxin B antibodies immobilized on an ultra thin platinum film sputtered onto a 100 μm thick silicon dioxide layer within a silicon chip. The film can be considered to be a collection of tiny capacitors connected in series and parallel over the film area. The impedance of this film is extremely sensitive to small changes in the electrical properties of the material between the enterotoxin B antibodies. Binding of enterotoxin B to enterotoxin B antibodies redistributes significant charges on the surface of the antibodies, which in turn decreases the observed impedance.

Similarly, U.S. Pat. No. 5,567,301 describes an immunobiosensor comprising an antibody covalently bound to a substrate material and a pair of electrodes. The biosensor is made by covalently binding the desired antibodies to an ultra-thin metal film sputtered onto a silicon chip. Further examples include the use of proteins immobilized on monomolecular alkylthiol films on gold electrodes [Mirsky et al., Biosens. Bioelectron. 12:977-989 (1997)]; a microfabricated biosensor chip that includes integrated detection elements and within which antibodies are attached to a capture surface (U.S. patent application Ser. No. 20010053535); and a sensor which uses an affinity component capable of interacting with analyte species and which is immobilized onto a conducting polymer such that the interaction between the affinity component and the analyte induces change in the electrical properties of the polymer (U.S. Pat. No. 6,300,123). Bioaffinity devices have also been described that are based on dipole moment changes [for example, see Hianik, T., et al., Biochem. Bioenerg., 47:47-55 (1998); Mulloni, V., et al, Physica Status Solidi, 182:479-484 (2000); DeSilva, M. S., et al., Biosensors & Bioelectronics, 10:675-682 (1995)].

The present invention, therefore, provides a biosensor comprising a CAD-glucokinase as the glucose sensor component. The biosensor can be incorporated into a hand-held device for conventional glucose monitoring, or into an implantable device as part of an open-loop system for continuous glucose monitoring. Alternatively, it can be incorporated into a closed-loop biomedical device for continuous glucose monitoring and insulin delivery. One skilled in the art will understand that a closed loop system can consist of a single unit comprising the biosensor and the insulin delivery system, or the biosensor and the insulin delivery system may constitute separate units. Advantages of separate units include optimal positioning of each unit, for example, the insulin delivery unit in the portal system and the glucose-sensing unit subcutaneously to facilitate access. The two units can be connected, for example, via a short telecommunications system utilising appropriate algorithms to dictate insulin delivery.

It will be readily understood by one skilled in the art that the CAD-glucokinase according to the present invention can be incorporated into various biosensor formats for use as a glucose sensor, including those devices described above and elsewhere. The field of biosensors and bioelectronic devices is rapidly evolving and new types of these devices are continuously being developed. The use of the CAD-glucokinase as a glucose sensor in both known and newly developed devices is therefore considered to be within the scope of the present invention.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

To gain a better understanding of the invention described herein, the following examples are set forth. It should be understood that these examples are for illustrative purposes only. Therefore, they should not limit the scope of this invention in any way.

### EXAMPLES

### Example 1

Cloning Human Glucokinase

The human liver glucokinase was cloned from the Hep 3B liver cell line. Following isolation of total mRNA from the cell line using standard techniques, RT-PCR was employed to generate sufficient glucokinase cDNA. Expand reverse transcriptase, a genetically engineered version of MoMuLV-RT that has negative RNase H activity, and an Oligo (dT) 15 primer were used for the reverse transcription step. PWO DNA Polymerase was used for the PCR step. PCR was performed in three separate reactions. The first reaction amplified a 5′ portion of the glucokinase cDNA, the second reaction amplified a 3′ portion of the glucokinase cDNA and the third reaction amplified the complete glucokinase sequence from the combined products of the first and second reactions. Primers were used that incorporated convenient restriction enzyme sites to facilitate cloning into appropriate vectors. Primers used to amplify the glucokinase for cloning into plasmid pcDNA3 (digested with BamHI and EcoRI) were:

| | |
|---|---|
| LGK-2 | |
| 5′-CCGGATCCAGATGGCGATGGATGTCACA-3′ | [SEQ ID NO:3] |
| SAC Ib | |
| 5′-GGTTTGCAGAGCTCTCGTCCAC-3′ | [SEQ ID NO:4] |
| SAC Ia | |
| 5′-GTGGACGAGAGCTCTGCAAACC-3′ | [SEQ ID NO:5] |
| GLK-3 | |
| 5′-CTGAATTCACTGGCCCAGCATACAG-3′ | [SEQ ID NO:6] |

Primers used to amplify the glucokinase for cloning into plasmid pGEX-KG (digested with Xho I and Hind III) were:

| | |
|---|---|
| LGK-3 | |
| 5′-CCCTCGAGATGGCGATGGATGTCACA-3′ | [SEQ ID NO:7] |
| SAC Ib | |
| 5′-GGTTTGCAGAGCTCTCGTCCAC-3′ | [SEQ ID NO:4] |
| SAC Ia | |
| 5′-GTGGACGAGAGCTCTGCAAACC-3′ | [SEQ ID NO:5] |
| GLK-3-2 | |
| 5′-CTAAGCTTACTGGCCCAGCATACAG-3′ | [SEQ ID NO:8] |

The final PCR products were cloned into pcDNA3 and pGEX-KG plasmids digested with the restriction enzymes indicated above and the inserts were sequenced.

pGEX-KG glucokinase clone 20 nucleotide sequence was confirmed to be the same as the wild-type sequence (i.e. human liver glucokinase 2; SEQ ID NO: 1). The glucokinase coding sequence from this clone was subcloned (using BamHI and HindIII restriction sites) into pcDNA3 (BamHI/EcoRV digested) to give pcDNA3 glucokinase clone 20. The glucokinase nucleotide sequence in both plasmid pGEX-KG glucokinase clone 20 and plasmid pcDNA3 glucokinase clone 20 is identical to the published sequence of the human liver glucokinase 2 cDNA (GenBank Accession Number M6905 1; SEQ ID NO:1).

### Example 2

Site-directed Mutagenesis of the Cloned Human Glucokinase

In vitro site-directed mutagenesis of the glucokinase was achieved by PCR-based techniques to create mutations at position 336 (Ser->Val; Ser->Leu and Ser->Ile) and at position 205 (Asp->Ala). The PCR reactions employed complementary primers containing mutagenic sequences, and a set of upstream and downstream primers. The sequences of the mutagenic primers were as follows (nucleotides that are different from those that occur in the wild type sequence are underlined):

| | |
|---|---|
| Ser336VaI | |
| Primer A | |
| 5′-TCGTGGTCCAGGTGGAGAGCG-3′ | [SEQ ID NO:9] |
| Primer B | |
| 5′-CGCTCTCCACCTGGACCACGA-3′ | [SEQ ID NO:10] |
| Ser336Leu | |
| Primer A | |
| 5′-TCGTGCTGCAGGTGGAGAGCG-3′ | [SEQ ID NO:11] |
| Primer B | |
| 5′-CGCTCTCCACCTGCAGCACGA-3′ | [SEQ ID NO:12] |
| Ser336Ile | |
| Primer A | |
| 5′-TCGTGATTCAGGTGGAGAGCG-3′ | [SEQ ID NO:13] |
| Primer B | |
| 5′-CGCTCTCCACCTGAATCACGA-3′ | [SEQ ID NO:14] |
| Asp205Ala | |
| Primer A | |
| 5′-GGTGAATGCAACGGTGGCCACG-3′ | [SEQ ID NO:15] |
| Primer B | |
| 5′-CGTGGCCACCGTTGCATTCACCC-3′ | [SEQ ID NO:16] |
| Primer GLK-3 | |
| 5′-CTGAATTCACTGGCCCAGCATACAG-3′ | [SEQ ID NO:6] |
| Primer 4A | |
| 5′-GACTTCCTGGACAAGCATCAGA-3′ | [SEQ ID NO:17] |

PCR products with overlapping sequences in which lie the implanted missense mutations were generated by three PCR reactions. All PCR reactions were performed using Vent DNA polymerase. For each mutant:

PCR Reaction 1) Primer 4A (upstream primer) and Primer A;

PCR Reaction 2) Primer B and Primer Glk 3 (downstream primer); and

PCR Reaction 3) mixture of products of PCR Reactions 1 and 2 with Primer A and Primer Glk 3.

The final PCR product for each mutant was digested with Sac II and BsrG1 and re-introduced into the pcDNA3 glucokinase clone (digested with Sac II and BsrG1).

### Example 3

Expression and Analysis of Wild-type and Mutant Glucokinases #1

The mutant glucokinase produced by the above PCR reactions were first cloned into pcDNA3 (as indicated above). Wild-type glucokinase and the mutant glucokinases were each subsequently subcloned into the expression vectors pGEX-KG and pET-15b using Xho I and BamH I restriction enzymes (blunt end ) for the wild-type and Sac II and BsrGI for the mutants.

The following plasmids were generated in this manner. All plasmids have been sequenced to confirm the presence of the appropriate mutant sequence and the absence of any abnormalities.

TABLE 1
List of Plasmids

| Plasmid | Clone # | Glucokinase |
|---|---|---|
| pGEX-KG | 20 | Wild-type |
| | 23 | Ser336Val |
| | 32 | Ser336Leu |
| | 43 | Ser336Ile |
| | 53 | Asp205Ala |
| pCDNA3 | 20 | Wild-type |
| | 7 | Ser336Val |
| | 15 | Ser336Leu |
| | 25 | Ser336Ile |
| | 33 | Asp205Ala |
| Pet-15b | 14 | Wild-type |
| | 1 | Ser336Val |
| | 9 | Ser336Leu |
| | 13 | Ser336Ile |
| | 18 | Asp205Ala |

Each mutant pcDNA3 clone was transfected into Cos cells using standard liposomal transfection methodology. On day 3 post transfection, glucokinase activity was measured (as described below in Example 4 [Trifiro, M. & Nathan, D., Prep. Biochem. 16:155-173, 1986]) and all mutant glucokinase proteins were shown to have null enzymes activity (i.e. below detectable limits).

### Example 4

Expression and Analysissis of Wild-type and Mutant Glucokinases #2

Wild-type and genetically engineered glucokinase are produced using the baculovirus high-level expression system. the wild-type glucokinase cDNA (liver and β-islet) is cloned into pBlueBacHis2 AcMNPV baculovirus plasmid. Each PCR in vitro mutagenesis experiment generating a genetically engineered, mutant glucokinase sequence is also directly cloned into pBlueBacHis2 AcMNPV wild-type glucokinase. When introduced into SF9 cells, some of these plasmids undergo a recombination event with co-transfected baculovirus genome and produce viral particles expressing glucokinase. Blue colonies represent successful expression due to concomitant β-gal expression. The plasmids also introduce a polyhistidine tag to the N-terminus of glucokinase, which allows for one-step purification of glucokinase from SF9 lysates using Ni²⁺ columns. The introduction of linearized AcMNPV DNA and smaller baculovirus vectors allows for high recombinant virus yield (˜80%) and easier subcloning. Yields are generally 10-100 μg glucokinase/ mg of SF9 lysate.

i) Analysis of Catalytic Activity

Glucokinase activity is assayed as described previously [Storer, A. C., et al., Biochem. J. 141:205-209

i) Analysis of Catalytic Activity (1974)] with modifications. Reactions are carried out at 25° C. in 50 nM glycylglycinate buffer, pH 8.0, containing 1 mM NADP, 100 mM KCl, 1 unit of glucose 6-P dehydrogenase, 100 mM glucose, 5 mM ATP, and 150 μl glucokinase in a total volume of 750 μl. Hexokinase is assayed under similar conditions except the final glucose concentration is 0.5 mM glucose. All reaction mixtures are incubated at 25° C. for 3 min. Hexokinase and glucose-6-P dehydrogenase activity are subtracted from the total activity observed with 100 mM glucose substrate to give the glucokinase activity.

When the glucokinase is assayed in the initial stages of purification, an excess of glucose-6-P dehydrogenase is present in the preparation. In order to take this into account, the recorded absorbance at 340 nm is divided by two when the activity of the homogenate is assayed.

Production of NADPH is followed by the increase in absorbance at 340 nm using a Beckman DU-6 spectrophotometer. Glucokinase activity is calculated according to the following formula:

Activity (units/ml)=(% OD/min/6.2)/0.15

A unit of activity is the amount of glucokinase which transforms, under optimal conditions, 1 μmole of substrate/min at room temperature.

ii) Analysis of Glucose and ATP Affinity

Both wild-type and mutant glucokinase are assessed for their ability to bind glucose and ATP. Preparations of wild-type and mutant glucokinase derived from glucokinase-expressing SF9 cells are immobilized on Ni²⁺ metal resin through the N-terminal polyhistidine tag. The binding experiments employ radiolabelled ³H-glucose and ³H-Mg-ATP (with constant specific activity) at various concentrations. The analysis is carried out in a “batch” technique, so as to simplify isolation and quantitation of bound and free counts of labelled substrates. Analysis is plotted as percent saturation plots vs. concentration (to ensure equilibrium end points) and as classical Scatchard analysis and is used to determine the affinity constants of the wild-type and mutant proteins for glucose and ATP.

ii) Dissociation Analysis

The ability of mutant glucokinase to release glucose or allow glucose to dissociate in a specific time frame will be an important issue. Thus, non-equilibrium dissociation constants (k_{d}) for glucose are determined for wild-type and for each mutant glucokinase.

Dissociation constants for wild-type and mutant glucokinase immobilized on Ni²⁺ affinity resin are determined by radiolabelling with ³H-glucose at a saturating concentration of glucose for an appropriate period of time followed by removal of ³H-glucose, and addition of 200× cold (unlabelled) glucose. This determination is conducted in a batch format. At various times, aliquots of the mixture are removed and the bound and free counts determined. The data obtained is plotted as % of substrate-glucokinase complexes remaining vs. time. Dissociation rates in such circumstances usually follow zero-order kinetics.

### Example 5

Conformational Analysis of the Wild-type and Genetically Engineered Glucokinase

The well documented large conformational change induced by glucose is key in pursuing a genetically engineered glucokinase as a glucose sensor. Thus, confirmation of such conformational change in the mutant glucokinases is needed. In order to determine the conformational change that is undergone by the wild-type glucokinase upon binding glucose, the phosphorylation reaction normally catalysed by the enzyme needs to be suppressed. The wild-type enzyme is, therefore, pre-treated with an appropriate ATP analogue, such as cibacron blue, basilen blue, suramin, TNP-ATP and ATP-α-S that will act as a suicide inhibitor and prevent phosphorylation and help retain glucose in the active site.

i) Partial Proteolytic Digestion

Partial trypsin digestion and the analysis of produced fragments by denaturing PAGE is used to demonstrate the large conformational change of wild-type glucokinase induced by glucose binding and to confirm that the mutant glucokinases undergo similar conformational change.

In vitro-transcribed and translated material can be used for this type of analysis and produces proteins which are radiolabelled to very high specific activity and which are immune to other contaminating radiolabelled proteins seen in the whole cell approach. In vitro transcribed and translated glucokinase has been shown to be enzymatically active thus retaining its quintessential native structure. Both the wild-type and mutant glucokinase cDNAs, therefore, are conjointly cloned into the pcDNA3 plasmid (the multiple cloning sites of the pcDNA3 and pBlueBacHis2 AcMNPV plasmids share common restriction enzymes). The Promega TNT system is used for same-tube coupled in vitro transcription/translation of the cDNAs. A chase of cold methionine is used to give a cleaner full-length protein product.

Alternatively, recombinant proteins are used for this experiment. Recombinant, labelled glucokinase and CAD-glucokinase are produced in high yields using the baculovirus expression system described above when radiolabelled methionine is included in the growth medium. The proteins are then purified on Ni²⁺-columns.

The proteins are then treated with trypsin and digests are allowed to run 0-10 minutes. All digests are then analyzed by 10% SDS-PAGE.

ii) Zero-order Cross-linking

Proteins for the zero-order cross-linking experiments are produced as described above. Prior to treatment with trypsin, the proteins are exposed to a tissue transglutaminase for an appropriate length of time [see, Safer, D., et al., Biochemistry, 36:5806-5816 (1997)]. Trypsin digests are either partial, as described above, or complete. Digests are analyzed by 10% SDS-PAGE.

iii) Measurement of Protein Dipole Moments

The dipole moments of the recombinant wild-type and CAD-glucokinase proteins are determined with the proteins in both the free and glucose-bound forms. Dipole moments are determined by dielectric relaxation spectroscopy employing frequency domain and/or time domain methodology [see Smith, G., et al., J. Pharm. Sci., 84:1029-1044 (1995)].

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

We claim:

1. A recombinant human glucokinase having decreased catalytic activity but a substantially identical ability to bind glucose relative to a corresponding wild-type human glucokinase.

2. The recombinant glucokinase according to claim 1 comprising a mutation at one or more residue selected from the group of: Asp78, Gly81, Thr82, Asn83, Arg85, Ser 151, Thr 168, Lys169, Asn204, Asp205, Thr228, Asn231, Glu256, Glu290, Lys296, Thr332, Ser336, Ser411 and Lys414.

3. The recombinant glucokinase according to claim 1, wherein said human glucokinase is the human liver glucokinase isoform 2.

4. The recombinant glucokinase according to claim 1, wherein said decreased catalytic activity is between about 10 and about 10 000-fold less than the catalytic activity of the corresponding wild-type human glucokinase.

5. The recombinant glucokinase according to claim 2, wherein said one or more mutation is at residue Asp205.

6. The recombinant glucokinase according to claim 2, wherein said one or more mutation is at residue Ser336.

7. The recombinant glucokinase according to claim 2, comprising mutations at residues Asp205 and Ser336.

8. The recombinant glucokinase according to claim 5, wherein the mutation is Asp205Ala.

9. The recombinant glucokinase according to claim 6, wherein the mutation is Ser336Val, Ser336Leu or Ser336Ile.

10. An isolated nucleic acid molecule encoding a mutant human glucokinase having decreased catalytic activity but a substantially identical ability to bind glucose relative to a corresponding wild-type human glucokinase.

11. The isolated nucleic acid according to claim 10, wherein said mutant glucokinase comprises a mutation at one or more residue selected from the group of: Asp78, Gly81, Thr82, Asn83, Arg85, Ser151, Thr 168, Lys169, Asn204, Asp205, Thr228, Asn231, Glu256, Glu290, Lys296, Thr332, Ser336, Ser411 and Lys414.

12. The isolated nucleic acid according to claim 10, wherein said human glucokinase is the human liver glucokinase isoform 2.

13. The isolated nucleic acid according to claim 11, wherein said mutation is at residue Asp205.

14. The isolated nucleic acid according to claim 11, wherein said mutation is at residue Ser336.

15. The isolated nucleic acid according to claim 11, comprising mutations at residues Asp205 and Ser336.

16. The isolated nucleic acid according to claim 13, wherein the mutation is Asp205Ala.

17. The isolated nucleic acid according to claim 14, wherein the mutation is Ser336Val, Ser336Leu or Ser336Ile.

18. A vector comprising the isolated nucleic acid molecule according to claim 10.

19. The vector according to claim 18, wherein said nucleic acid molecule is operably linked to a regulatory control sequence.

20. A host cell comprising the vector according to claim 18.

21. A method of producing a recombinant human glucokinase comprising:
(a) culturing the host cell according to claim 20 under conditions in which the encoded glucokinase is expressed; and
(b) isolating the expressed glucokinase.

22. A glucose sensor comprising a recombinant human glucokinase having decreased catalytic activity but a substantially identical ability to bind glucose relative to the corresponding wild-type human glucokinase.

23. The glucose sensor according to claim 22, wherein said recombinant glucokinase is immobilised on a solid support.

24. The glucose sensor according to claim 22, wherein said glucose sensor is implantable.

25. A method of determining the level of glucose in a sample comprising:
(a) contacting said sample with a recombinant human glucokinase having decreased catalytic activity but a substantially identical ability to bind glucose relative to a corresponding wild-type human glucokinase;
(b) measuring a change in a physical characteristic of said recombinant glucokinase; and
(c) correlating said change to the level of glucose in said sample.

26. The method according to claim 25, wherein said contacting and said measuring are conducted in vivo.

27. The method according to claim 25, wherein said measuring is conducted on a continuous basis thereby providing a continuous determination of the level of glucose in said sample.


SEQUENCE LISTING (17 sequences)

SEQ ID NO: 1 - DNA, 2550 bp, Homo sapiens
  misc_feature (286)...(1680): human liver glucokinase 2 cDNA
aagccctgggctgccagcctcaggcagctctccatccaagcagccgttgctgccacaggcgggccttacgctccaaggctacagcatgtgctaggcctcagcaggcaggagcatctctgcctcccaaagcatctacctcttagcccctcggagagatggcgatggatgtcacaaggagccaggcccagacagccttgactctgccagactctcctctgaactcgggcctcacatggccaactgctacttggaacaaatcgccccttggctggcagatgtgttaacatgcccagaccaagatcccaactcccacaacccaactcccaggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagacgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgagagcagtggccgcaagcgcagggaggatgccacagccccacagcacccaggctccatggggaagtgctccccacacgtgctcgcagcctggcggggcaggaggcctggccttgtcaggacccaggccgcctgccataccgctggggaacagagcgggcctcttccctcagtttttcggtgggacagccccagggccctaacgggggtgcggcaggagcaggaacagagactctggaagccccccacctttctcgctggaatcaatttcccagaagggagttgctcactcaggactttgatgcatttccacactgtcagagctgttggcctcgcctgggcccaggctctgggaaggggtgccctctggatcctgctgtggcctcacttccctgggaactcatcctgtgtggggaggcagctccaacagcttgaccagacctagacctgggccaaaagggcaggccaggggctgctcatcacccagtcctggccattttcttgcctgaggctcaagaggcccagggagcaatgggagggggctccatggaggaggtgtcccaagctttgaataccccccagagaccttttctctcccataccatcactgagtggcttgtgattctgggatggaccctcgcagcaggtgcaagagacagagcccccaagcctctgccccaaggggcccacaaaggggagaagggccagccctacatcttcagctcccatagcgctggctcaggaagaaaccccaagcagcattcagcacaccccaagggacaaccccatcatatgacatgccaccctctccatgcccaacctaagattgtgtgggttttttaattaaaaatgttaaaagttttaaaaaaaaaa

SEQ ID NO: 2 - protein, 464 aa, Homo sapiens [sequence omitted]

SEQ ID NO: 3 - DNA, 28 bp, Artificial Sequence
  Description of Artificial Sequence LGK-2 primer
ccggatccagatggcgatggatgtcaca

SEQ ID NO: 4 - DNA, 22 bp, Artificial Sequence
  Description of Artificial Sequence SAC Ib primer
ggtttgcagagctctcgtccac

SEQ ID NO: 5 - DNA, 22 bp, Artificial Sequence
  Description of Artificial Sequence AC Ia primer
gtggacgagagctctgcaaacc

SEQ ID NO: 6 - DNA, 25 bp, Artificial Sequence
  Description of Artificial Sequence LK-3 primer
ctgaattcactggcccagcatacag

SEQ ID NO: 7 - DNA, 26 bp, Artificial Sequence
  Description of Artificial Sequence GK-3 primer
ccctcgagatggcgatggatgtcaca

SEQ ID NO: 8 - DNA, 25 bp, Artificial Sequence
  Description of Artificial Sequence LK-3-2 primer
ctaagcttactggcccagcatacag

SEQ ID NO: 9 - DNA, 21 bp, Artificial Sequence
  Description of Artificial Sequence er336Val primer A
tcgtggtccaggtggagagcg

SEQ ID NO: 10 - DNA, 21 bp, Artificial Sequence
  Description of Artificial Sequence er336Val primer B
cgctctccacctggaccacga

SEQ ID NO: 11 - DNA, 21 bp, Artificial Sequence
  Description of Artificial Sequence er336Leu primer A
tcgtgctgcaggtggagagcg

SEQ ID NO: 12 - DNA, 21 bp, Artificial Sequence
  Description of Artificial Sequence er336Leu primer B
cgctctccacctgcagcacga

SEQ ID NO: 13 - DNA, 21 bp, Artificial Sequence
  Description of Artificial Sequence er336Ile primer A
tcgtgattcaggtggagagcg

SEQ ID NO: 14 - DNA, 21 bp, Artificial Sequence
  Description of Artificial Sequence er336Ile primer B
cgctctccacctgaatcacga

SEQ ID NO: 15 - DNA, 22 bp, Artificial Sequence
  Description of Artificial Sequence sp205Ala primer A
ggtgaatgcaacggtggccacg

SEQ ID NO: 16 - DNA, 23 bp, Artificial Sequence
  Description of Artificial Sequence sp205Ala primer B
cgtggccaccgttgcattcaccc

SEQ ID NO: 17 - DNA, 22 bp, Artificial Sequence
  Description of Artificial Sequence rimer 4A
gacttcctggacaagcatcaga